(19) United States
(12) Patent Application Publication
(10) Pub. No.: US 2023/0227878 A1
(43) Pub. Date: Jul. 20, 2023

(54) PROCESS FOR PREPARING SURFACTANT

(71) Applicants: Enza Biotech AB, Lund (SE); Croda International Plc, Goole, East Yorkshire (GB)

(72) Inventors: Stefan Ulvenlund, Lund (SE); Maria Andersson, Lund (SE); Maria Viloria Cols, Lund (SE); Nikolina Barchan, Lund (SE); Christian Wells, Goole, East Yorkshire (GB); Johan Sander, Lund (SE); Magdalena Axenstrand, Lund (SE)

(73) Assignees: Enza Biotech AB, Lund (SE); Croda International Plc, Goole, East Yorkshire (GB)

(21) Appl. No.: 18/010,361

(22) PCT Filed: Jun. 18, 2021
(86) PCT No.: PCT/EP2021/066631

§ 371 Date: Dec. 14, 2022

(30) Foreign Application Priority Data
Jun. 19, 2020 (GB) 2009392.8

(51) Int. Cl. C12P 19/18; C12P 19/04

(52) U.S. Cl. CPC C12P 19/18; C12P 19/04

(57) ABSTRACT

A process for the preparation of C4 to C24 alkyl polyglycosides by the use of an enzyme to react C4 to C24 alkyl glycoside with a glycosyl donor containing monosaccharide residues, wherein the C4 to C24 alkyl polyglycosides have a mole-average degree of polymerization (mean DP) of the glycoside chains of greater than 1.5 units. The C4 to C24 alkyl polyglycosides are particularly useful in personal care formulations.

### FIELD OF INVENTION

The present invention relates to a process for the preparation of alkyl polyglycosides by an enzyme reaction, alkyl polyglycoside compositions per se and use thereof.

### BACKGROUND

Alkyl glycoside, particularly alkyl polyglycoside, and especially alkyl polyglucoside, non-ionic surfactants have been widely used in a range of cosmetic, household, health care and industrial applications. Existing commercially available alkyl polyglucosides are produced by a chemical route. Methods of producing alkyl polyglycosides by use of an enzyme reaction have been disclosed in the literature, but at present no suitable commercially viable method exists for the enzymatic synthesis of alkyl polyglycosides such as alkyl polyglucosides. The enzyme reaction also results in a reaction mixture containing residual sugars such as linear oligosaccharides and cyclodextrin (some of which may be original starting material), which can be difficult to separate from the alkyl polyglycosides. There is a need to improve the efficiency and/or yield of the enzyme reaction, and to reduce the quantity of sugars, particularly cyclodextrin, in the enzyme reaction product mixture.

Commercially available alkyl polyglycosides are mixtures of molecules wherein the mean length of the polyglycoside chain is short, despite being commonly referred to as “poly”, varying from about 1 to 1.5 glycoside units, preferably glucose, units per alkyl chain. This limits the usefulness of the surfactants and there is a need for alkyl polyglycosides, particularly alkyl polyglucosides, with longer glycoside/glucoside chains. There is also a need to be able to vary the distribution of the glycoside chains in the mixture to modify/improve the surfactant properties of the alkyl polyglycoside. Some of these properties are difficult to achieve using chemical synthesis methods.

### SUMMARY OF THE INVENTION

We have surprisingly discovered a process of preparing alkyl polyglycosides by an enzyme reaction, alkyl polyglycoside compositions per se and use thereof, which overcomes or significantly reduces at least one of the aforementioned problems.

Accordingly, the present invention provides a process for the preparation of C4 to C24 alkyl polyglycosides by reacting with an enzyme a C4 to C24 alkyl glycoside and a glycosyl donor comprising monosaccharide residues, wherein (a) the reaction mixture comprises (i) monosaccharide residues in the glycosyl donor to alkyl glycoside at a molar ratio of less than 40.0:1.0, and optionally (ii) alkyl glycoside at greater than or equal to 1.0 wt %; (b) greater than or equal to 3.0 wt % of the monosaccharide residues in the glycosyl donor are transferred to the alkyl glycoside (glycoside units conversion); and (c) the reaction product comprises alkyl polyglycoside optionally comprising (i) greater than 0.10 mole fraction of alkyl monoglycoside (DP1), and/or (ii) a mole-average degree of polymerization (mean DP) of the glycoside chains of greater than or equal to 1.5 units.

The invention further provides a process of reacting with an enzyme;

(i) a glycosyl donor comprising monosaccharide residues; and
(ii) an alkyl glycoside of the formula Rₘ-Gₙ, wherein
R is an alkyl group comprising m carbon atoms,
m is 4 to 24,
G is at least one monosaccharide residue, and
n is the number of monosaccharide residues;
in a reaction mixture to form a reaction product comprising;
(iii) an alkyl polyglycoside of the formula Rₚ-G_{q},
wherein
R is an alkyl group comprising p carbon atoms, p is 4 to 24,
G is at least one monosaccharide residue,
q is the number of monosaccharide residues, and the mean value of q is greater than or equal to 1.5,
q=(n+s) wherein n is defined in (ii) and s is the increase in the number of monosaccharide residues that occurs during the enzyme reaction, and
the mean value of s is greater than or equal to 0.5; and
(iv) greater than or equal to 3.0 wt % of the monosaccharide residues in the glycosyl donor are transferred to the alkyl glycoside (glycoside units conversion) during the enzyme reaction.

The invention also provides a composition comprising C4 to C24 alkyl polyglycosides wherein the amount of alkyl monoglycoside (DP1) is greater than 0.10 mole fraction and the mole-average degree of polymerization (mean DP) of the glycoside chains is greater than or equal to 1.8 units.

The invention yet further provides enzymatically produced C4 to C24 alkyl polyglycosides wherein the amount of alkyl monoglycoside (DP1) is greater than 0.10 mole fraction and the mole-average degree of polymerization (mean DP) of the glycoside chains is greater than or equal to 1.5 units.

The invention still further provides C4 to C24 alkyl polyglycosides obtainable by enzyme reaction wherein the amount of alkyl monoglycoside (DP1) is greater than 0.10 mole fraction and the mole-average degree of polymerization (mean DP) of the glycoside chains is greater than or equal to 1.8 units.

The invention also further provides the use of a C4 to C24 alkyl polyglycoside composition, wherein the mole-average degree of polymerization (mean DP) of the glycoside chains is greater than or equal to 1.8 units, and optionally the amount of alkyl monoglycoside (DP1) is greater than 0.10 mole fraction, to solubilize an active ingredient.

The invention even further provides the use of a C4 to C24 alkyl polyglycoside composition, wherein the mole-average degree of polymerization (mean DP) of the glycoside chains is greater than or equal to 1.8 units, and optionally the amount of alkyl monoglycoside (DP1) is greater than 0.10 mole fraction, to partially or completely replace polysorbate and/or alkyl glycoside in a personal care or health care formulation.

The alkyl glycoside starting material for use in the process of the present invention may be an alkyl monoglycoside, an alkyl diglycoside, an alkyl oligoglycoside and/or an alkyl polyglycoside. The glycoside component of the alkyl glycoside is suitably a monosaccharide residue, e.g. of glucose, fructose, mannose, galactose, arabinose, and mixtures thereof, and/or one or more of these monosaccharide residues joined by glycosidic linkages, e.g. to form disaccharide, oligosaccharide and/or polysaccharide chains. The monosaccharide residues suitably comprise, consist essentially of, or consist of glucose residues. Thus, preferred starting materials are alkyl glucosides selected from the group consisting of an alkyl monoglucoside, an alkyl diglucoside, an alkyl oligoglucoside, an alkyl polyglucoside, and mixtures thereof, more preferably from an alkyl monoglucoside, an alkyl diglucoside, an alkyl oligoglucoside, and mixtures thereof, and particularly from an alkyl monoglucoside and an alkyl diglucoside, for example an alkyl maltoside, and mixtures thereof.

In one embodiment, the alkyl glycoside starting material will be a composition containing a mixture of compounds, e.g. comprising different alkyl and/or glycoside chains. Commercially available alkyl glycosides, preferably alkyl glucosides, may be used as starting materials. Some commercially available mixtures of alkyl glycosides are generally referred to as alkyl polyglycosides or alkyl polyglucosides even though the mean length or mole-average degree of polymerization (mean DP) of the glycoside/glucoside chains will generally be less than 1.5 units.

For the avoidance of doubt, the use of the terms “alkyl glycosides” and “alkyl glucosides” herein shall generally refer to the starting materials for the enzyme reaction, unless otherwise clear from the context. The products, i.e. compositions or mixtures, resulting from the enzyme reaction shall be referred to herein as “alkyl polyglycosides” and/or “alkyl polyglucosides”.

The alkyl chain of the alkyl glycoside, preferably alkyl glucoside, may be linear or branched, preferably comprises, consists essentially of, or consists of linear chains. The length or number of carbon atoms in the alkyl chain suitably comprises, consists essentially of, or consists of in the range from C4 to C24, preferably C8 to C20, more preferably C10 to C18, particularly C10 to C16, and especially C12 and C14.

In one embodiment, the alkyl glycoside may be solely in the α-anomer or β-anomer form, but may comprise both anomers, suitably having an α:β anomer ratio in the range from 0.01 to 100:1.0, preferably 0.05 to 20.0:1.0, more preferably 0.1 to 10.0:1.0, particularly 0.3 to 3.5:1.0, and especially 0.5 to 2.0:1.0.

In one embodiment, the molar mean length or mean number of carbon atoms of the alkyl chain of the alkyl glycoside, preferably alkyl glucoside, groups are in the range from 4.0 to 24.0, preferably 6.0 to 20.0, more preferably 8.0 to 17.0, particularly 10.0 to 16.0, and especially 12.0 to 14.0.

In one embodiment, the alkyl chain of the alkyl glycoside, preferably alkyl glucoside, comprises, consists essentially of, or consists of a mixture of C12 and C14 alkyl groups, suitably wherein the molar ratio of C12:C14 alkyl groups is in the range from 0.5 to 15:1.0, preferably 1.5 to 8:1.0, more preferably 2.0 to 4.0:1.0, particularly 2.6 to 3.4:1.0, and especially 2.9 to 3.1:1.0.

In one embodiment, the alkyl chain of the alkyl glycoside, preferably alkyl glucoside, comprises, consists essentially of, or consists of a mixture of C8 and 010 alkyl groups, suitably wherein the molar ratio of C8:C10 alkyl groups is in the range from 0.1 to 10.0:1.0, preferably 0.3 to 3.5:1.0, more preferably 0.5 to 2.0:1.0, and particularly 0.8 to 1.3:1.0.

In one embodiment, the alkyl chain of the alkyl glycoside, preferably alkyl glucoside, comprises, consists essentially of, or consists of a mixture of C8, C10, C12 and C14 alkyl groups.

In one embodiment, the alkyl chain of the alkyl glycoside, preferably alkyl glucoside, comprises, consists essentially of, or consists of a mixture of C8, C10, C12, C14 and C16 alkyl groups.

In one embodiment, the alkyl chain of the alkyl glycoside, preferably alkyl glucoside, comprises, consists essentially of, or consists of a mixture of C12, C14 and C16 alkyl groups.

In one embodiment, the alkyl chain of the alkyl glycoside, preferably alkyl glucoside, may be selected from the group consisting of lauryl glucoside, decyl glucoside, caprylyl/capryl glucoside, coco-glucoside and mixtures thereof.

In one embodiment, the alkyl glycoside comprises, consists essentially of, or consists of an alkyl monoglycoside and/or an alkyl diglycoside, preferably alkyl monoglucoside and/or alkyl diglucoside, particularly C12 and/or C14 alkyl glucosides, and especially C12 and C14 alkyl glucosides.

In one embodiment, the alkyl glycoside, preferably alkyl glucoside, starting material preferably comprises (i) greater than or equal to 80 wt %, more preferably in the range from 85 to 99 wt %, particularly 90 to 97 wt %, and especially 94 to 96 wt % of alkyl monoglycoside and/or (ii) less than or equal to 20 wt %, more preferably in the range from 1 to 15 wt %, particularly 3 to 10 wt %, and especially 4 to 6 wt % of alkyl diglycoside, both based on the total weight of alkyl glycoside.

In one embodiment, the alkyl glycoside, preferably alkyl glucoside, comprises a mixture of compounds wherein the mean DP of the glycoside chain is suitably in the range from 1.0 to 1.7, preferably 1.0 to 1.5, more preferably 1.0 to 1.3, particularly 1.0 to 1.15, and especially 1.0 to 1.1 glycoside units, preferably glucose units, per alkyl chain.

The alkyl glycoside starting material can be represented by the formula Rₘ-Gₙ, wherein

R is an alkyl group comprising m carbon atoms, both defined herein,
G is at least one monosaccharide residue, defined herein,
n is the number of monosaccharide residues, and the mean value of n is defined herein (mean DP).

The glycosyl donor starting material is suitably a cyclic, linear or branched oligosaccharide or polysaccharide, or mixture thereof. The glycosyl donor may comprise a cyclic carbohydrate, i.e. a carbohydrate in which the chain of monosaccharide residues forms a closed loop (such as α-, β-, γ-cyclodextrin or larger cyclic alpha-glucans), linear oligosaccharides, such as maltodextrin, and polysaccharides, such as starch etc.

In one preferred embodiment, the glycosyl donor is selected from the group consisting of maltodextrin, cyclodextrin, starch and mixtures thereof; preferably maltodextrin, cyclodextrin and mixtures thereof; more preferably maltodextrin or cyclodextrin; and particularly cyclodextrin.

In one embodiment, the glycosyl donor comprises, consists essentially of, or consists of α-, β-, and/or γ-cyclodextrin, preferably α- and/or β-cyclodextrin, more preferably β-cyclodextrin.

In one embodiment, the glycosyl donor comprises, consists essentially of, or consists of starch, particularly waxy starch. The starch may be derived from any vegetable source, e,g. corn, wheat, maize, barley, potato, tapioca, rice, sago, and sorghum grain. Crude starch materials such as ground cereals, macerated tubers, or the partially purified starches therefrom may be used. The term “starch” used herein encompasses unmodified starch as well as starch that has been modified by treatment with acids, alkalies, enzymes, heat, etc. Soluble or partially soluble modified starches, dextrins, pregelatinized products and starch derivatives of different types may also be used as glycosyl donor. Waxy (i.e. high in amylopectin) starch is preferred, such as those selected from the group consisting of potato amylopectin, maize amylopectin, waxy maize starch, waxy barley starch, waxy potato starch, and mixtures thereof.

In one embodiment, the glycosyl donor comprises, consists essentially of, or consists of maltodextrin. The maltodextrin may be derived from any vegetable source, such as potato, maize and wheat. Potato maltodextrin is one preferred form.

In one embodiment, the dextrose equivalent (DE) value of the maltodextrin is suitably in the range from 0.1 to 20, preferably 0.5 to 10, more preferably 0.8 to 5, particularly 0.9 to 2, and especially 1 to 1.5 units.

The enzyme used in the process of the present invention is capable of transferring at least one, preferably at least two, monosaccharide residues at a time from the glycosyl donor to the alkyl glycoside. The enzyme is preferably a glycoside (or glycosyl) hydrolase and/or glycoside transferase.

In one embodiment, the enzyme is a glycoside hydrolase or a glycosyl transferase, preferably a glycoside hydrolase, particularly belonging to glycoside hydrolase family 13 or 57. One preferred glycoside hydrolase family 13 enzyme is cyclodextrin glycosyltransferase, which is also known as cyclodextrin glucanotransferase or cyclodextrin glucanyltransferase or cyclodextrin glycosyltransferase (all abbreviated to CGTase). One preferred CGTase enzyme is a cyclomaltodextrin glucanotransferase (EC number 2.4.1.19) ((I-4)-alpha-D-glucan: (I-4)-alpha-D-glucan 4-alpha-D[(I-4)-alpha-D-glucano]-transferase). Suitable enzymes include Bacillus macerans CGTase (Amano Enzyme Europe, U.K.) and Thermoanaerobacter sp. CGTase (Novozymes AJS, Denmark).

Other suitable glycoside hydrolase enzymes classified under family 13 and family 57, include 4-alpha-glucanotranserase, EC number 2.4.1.25; systematic name: (I-4)-alpha-D-glucan: (I-4)-alpha-D-glucan 4-alpha-D-glycosyl transferase (GTase).

In addition, glycosyl hydrolases belonging to other families or glycosyl transferases can be used in the process of the invention provided that they can transfer at least one, preferably at least two, monosaccharide residue at a time from the glycosyl donor to the alkyl glycoside as described herein.

In one embodiment, the monosaccharide residues present in the glycosyl donor are in molar excess compared to the alkyl glycoside in the reaction mixture of the process of the present invention. Suitably the molar ratio of monosaccharide residues present in the glycosyl donor, preferably maltodextrin, to alkyl glycoside in the reaction mixture is (i) greater than 2.0:1.0, preferably greater than or equal to 4.0:1.0, more preferably greater than or equal to 6.0:1.0, particularly greater than or equal to 8.0:1.0, and especially greater than or equal to 10.0:1.0; and/or (ii) less than 40.0:1.0, preferably less than or equal to 35.0:1.0, more preferably less than or equal to 30.0:1.0, particularly less than or equal to 25.0:1.0, and especially less than or equal to 20.0:1.0.

In one embodiment, the molar ratio of monosaccharide residues present in the glycosyl donor, preferably maltodextrin, to alkyl glycoside in the reaction mixture is suitably in the range from 11.0 to 19.0:1.0, preferably 12.0 to 18.0:1.0, more preferably 13.0 to 17.0:1.0, particularly 13.5 to 16.0:1.0, and especially 14.0 to 15.0:1.0.

In one embodiment, the molar ratio of monosaccharide residues present in the glycosyl donor, preferably cyclodextrin, to alkyl glycoside in the reaction mixture is (i) suitably greater than or equal to 0.8:1.0, preferably greater than or equal to 1.5:1.0, more preferably greater than or equal to 2.0:1.0, particularly greater than or equal to 2.5:1.0, and especially greater than or equal to 3.0:1.0; and/or (ii) suitably less than 20.0:1.0, preferably less than or equal to 15.0:1.0, more preferably less than or equal to 10.0:1.0, particularly less than or equal to 8.0:1.0, and especially less than or equal to 6.0:1.0.

In one embodiment, the molar ratio of monosaccharide residues present in the glycosyl donor, preferably cyclodextrin, to alkyl glycoside in the reaction mixture is suitably in the range from 3.1 to 5.7:1.0, preferably 3.3 to 5.0:1, more preferably 3.5 to 4.6:1.0, particularly 3.7 to 4.3:1.0, and especially 3.9 to 4.1:1.0.

In one embodiment, the molar ratio of monosaccharide residues present in the glycosyl donor, preferably β-cyclodextrin, to alkyl glycoside in the reaction mixture is suitably in the range from 0.8 to 2.5:1.0, preferably 1.2 to 2.2:1, more preferably 1.4 to 1.9:1.0, particularly 1.5 to 1.7:1.0, and especially 1.55 to 1.65:1.0.

In one embodiment, the weight ratio of glycosyl donor, preferably maltodextrin, to alkyl glycoside in the reaction mixture, i.e. used in the process of the present invention is suitably in the range from 2.0 to 15.0:1.0, preferably 4.0 to 10.0:1.0, more preferably 5.0 to 8.0:1.0, particularly 5.5 to 7.0:1.0, and especially to 6.0 to 6.4:1.0.

In one embodiment, the weight ratio of glycosyl donor, preferably cyclodextrin, to alkyl glycoside in the reaction mixture, i.e. used in the process of the present invention is suitably in the range from 0.8 to 3.0:1.0, preferably 1.2 to 2.5:1.0, more preferably 1.5 to 2.0:1.0, particularly 1.6 to 1.9:1.0, and especially to 1.7 to 1.8:1.0.

In one embodiment, the weight ratio of glycosyl donor, preferably β-cyclodextrin, to alkyl glycoside in the reaction mixture, i.e. used in the process of the present invention is suitably in the range from 0.2 to 1.5:1.0, preferably 0.4 to 1.0:1.0, more preferably 0.55 to 0.85:1.0, particularly 0.6 to 0.8:1.0, and especially to 0.65 to 0.75:1.0.

In one embodiment, the concentration of alkyl glycoside in the reaction mixture is (i) suitably greater than or equal to 1.0 wt %, preferably greater than or equal to 3.0 wt %, more preferably greater than or equal to 5.0 wt %, particularly greater than or equal to 7.0 wt %, and especially greater than or equal to 8.0 wt %; and/or (ii) suitably less than or equal to 30.0 wt %, preferably less than or equal to 20.0 wt %, more preferably less than or equal to 15.0 wt %, particularly less than or equal to 13.0 wt %, and especially less than or equal to 12.0 wt %, both based on the total weight of the mixture.

In one embodiment, the concentration of alkyl glycoside in the reaction mixture, preferably when maltodextrin is the glycosyl donor, is suitably in the range from 3.0 to 7.0 wt %, preferably 3.5 to 6.5 wt %, more preferably 4.0 to 6.0 wt %, particularly 4.3 to 5.7 wt %, and especially 4.5 to 5.5 wt %, based on the total weight of the mixture.

In one embodiment, the concentration of alkyl glycoside in the reaction mixture, preferably when cyclodextrin is the glycosyl donor, is suitably in the range from 7.5 to 12.5 wt %, preferably 8.0 to 12.0 wt %, more preferably 8.5 to 11.5 wt %, particularly 9.0 to 11.0 wt %, and especially 9.5 to 10.5 wt %, based on the total weight of the mixture.

In one embodiment, the concentration of alkyl glycoside in the reaction mixture, preferably when β-cyclodextrin is the glycosyl donor, is suitably in the range from 10.0 to 30.0 wt %, preferably 15.0 to 28.0 wt %, more preferably 20.0 to 26.0 wt %, particularly 21.0 to 25.0 wt %, and especially 22.0 to 24.0 wt %, based on the total weight of the mixture.

In one embodiment, the concentration of glycosyl donor, preferably maltodextrin, in the reaction mixture is greater than or equal to 10.0 wt %, suitably in the range from 10.0 to 55.0 wt %, preferably 15.0 to 45.0 wt %, more preferably 25.0 to 38.0 wt %, particularly 28.0 to 34.0 wt %, and especially 30.0 to 32.0 wt %%, based on the total weight of the mixture.

In one embodiment, the concentration of glycosyl donor, preferably cyclodextrin, in the reaction mixture is greater than or equal to 5.0 wt %, suitably in the range from 5.0 to 35.0 wt %, preferably 10.0 to 25.0 wt %, more preferably 15.0 to 20.0 wt %, particularly 16.0 to 19.0 wt %, and especially 17.0 to 18.0 wt %%, based on the total weight of the mixture.

In one embodiment, the amount of water in the reaction mixture is suitably in the range from 20.0 to 90.0 wt %, preferably 30.0 to 85.0 wt %, more preferably 40.0 to 80.0 wt %, particularly 50.0 to 75.0 wt %, and especially 60.0 to 70.0 wt %, based on the total weight of the mixture.

The concentration of enzyme in the reaction mixture is suitably in the range from 0.001 to 3.0 wt %, preferably 0.01 to 1.5 wt %, more preferably 0.1 to 1.0 wt %, particularly 0.3 to 0.7 wt %, and especially 0.4 to 0.6 wt %, based on the total weight of the mixture.

In one embodiment, the activity of the enzyme per kg of reaction mixture is suitably in the range from 1.0 to 60, preferably 5.0 to 50, more preferably 10 to 45, particularly 13 to 40, and especially 15 to 35 KNU-CP.

In one embodiment, the process of the present invention, preferably when maltodextrin is the glycosyl donor, is suitably carried out at a temperature in the range from 40 to 80° C., preferably 50 to 74° C., more preferably 55 to 71° C., particularly 60 to 69° C., and especially 63 to 67° C.

In one embodiment, the process of the present invention, preferably when cyclodextrin, particularly β-cyclodextrin, is the glycosyl donor, is suitably carried out at a temperature in the range from 65 to 85° C., preferably 70 to 80° C., more preferably 72 to 78° C., particularly 74 to 76° C., and especially 75° C.

In one embodiment, the enzyme reaction preferably occurs at a pH in the range from 5.0 to 9.0, more preferably 5.5 to 8.0, particularly 5.8 to 7.0, and especially 6.0 to 6.5.

The enzyme reaction suitably occurs over a time period in the range from 1 to 72, preferably 3 to 48, more preferably 5 to 36, particularly 6 to 24, and especially 10 to 20 hours. The enzyme reaction is suitably stopped after this time period by inactivation of the enzyme, for example, by heat, or by addition of acid, base or other agents. or by removal of the enzyme from the reaction mixture. In one embodiment, the enzyme is inactivated by heating the reaction mixture up to 100° C., suitably to 70° C., preferably to 80° C., more preferably to 85° C., particularly to 90° C., and especially to 95° C. for a suitable time period, e.g. for up to 2 hours, preferably up to 3 hours.

One advantage of the process of the present invention is that surprisingly high levels of glycoside units, preferably glucose, conversion or transfer of monosaccharide residues present in the glycosyl donor to the alkyl glycoside starting material can occur. The level of glycoside units conversion for the process of the present invention is defined as the weight percentage of the monosaccharide residues present in the glycosyl donor starting material which have been consumed or transferred by the enzyme to form the alkyl polyglycoside reaction product. The level of glycoside units conversion of the enzyme reaction according to the present invention can be determined by the HPLC analysis of the reaction products, as described herein.

In one embodiment, the level of glycoside, preferably glucose, units conversion or amount of monosaccharide residues present in the glycosyl donor, preferably maltodextrin, transferred to the alkyl glycoside starting material during the enzyme reaction is (i) suitably greater than or equal to 3.0 wt %, preferably greater than or equal to 7.0 wt %, more preferably greater than or equal to 9.0 wt %, particularly greater than or equal to 10.0 wt %, and especially greater than or equal to 11.0 wt %; and/or (ii) suitably less than or equal to 35.0 wt %, preferably less than or equal to 30.0 wt %, more preferably less than or equal to 25.0 wt %, particularly less than or equal to 20.0 wt %, and especially less than or equal to 15.0 wt %, both based on the weight of monosaccharide residues originally present in the glycosyl donor starting material.

In one embodiment, the level of glycoside, preferably glucose, units conversion in the process of the present invention, preferably when using maltodextrin as glycosyl donor, is suitably in the range from 10.0 to 15.0 wt %, preferably 10.5 to 14.0 wt %, more preferably 11.0 to 13.5 wt %, particularly 11.5 to 13.0 wt %, and especially 12.0 to 12.5 wt %, based on the weight of monosaccharide residues originally present in the glycosyl donor starting material.

In one embodiment, the level of glycoside, preferably glucose, units conversion in the process of the present invention, preferably when using cyclodextrin as glycosyl donor, is (i) suitably greater than or equal to 15.0 wt %, preferably greater than or equal to 20.0 wt %, more preferably greater than or equal to 25.0 wt %, particularly greater than or equal to 30.0 wt %, and especially greater than or equal to 35.0 wt %; and/or (ii) suitably less than or equal to 75.0 wt %, preferably less than or equal to 70.0 wt %, more preferably less than or equal to 65.0 wt %, particularly less than or equal to 60.0 wt %, and especially less than or equal to 55.0 wt %, both based on the weight of monosaccharide residues originally present in the glycosyl donor starting material.

In one embodiment, the level of glycoside, preferably glucose, units conversion in the process of the present invention, particularly when using cyclodextrin as glycosyl donor, is suitably in the range from 36.0 to 54.0 wt %, preferably 38.0 to 52.0 wt %, more preferably 40.0 to 50.0 wt %, particularly 42.0 to 48.0 wt %, and especially 44.0 to 46.0 wt %, based on the weight of monosaccharide residues originally present in the glycosyl donor starting material.

In one embodiment, the level of glycoside, preferably glucose, units conversion in the process of the present invention, preferably when using β-cyclodextrin as glycosyl donor, is greater than or equal to 15.0 wt %, suitably greater than or equal to 25.0 wt %, preferably greater than or equal to 35.0 wt %, more preferably greater than or equal to 45.0 wt %, particularly greater than or equal to 50.0 wt %, and especially greater than or equal to 55.0 wt %.

In one embodiment, the level of glycoside, preferably glucose, units conversion in the process of the present invention, particularly when using β-cyclodextrin as glycosyl donor, is suitably in the range from 45.0 to 75.0 wt %, preferably 50.0 to 70.0 wt %, more preferably 55.0 to 65.0 wt %, particularly 58.0 to 62.0 wt %, and especially 59.0 to 61.0 wt %, based on the weight of monosaccharide residues originally present in the glycosyl donor starting material.

In one embodiment, the concentration of alkyl polyglycoside in the crude reaction product mixture, i.e. before any purification or separation steps, is suitably in the range from 3.0 to 40.0 wt %, preferably 5.0 to 25.0 wt %, more preferably 7.0 to 21.0 wt %, particularly 8.0 to 19.0 wt %, and especially 8.5 to 18.0 wt %, based on the total weight of the mixture.

In one embodiment, the concentration of alkyl polyglycoside in the crude reaction product mixture is suitably in the range from 6.0 to 14.0 wt %, preferably 7.0 to 12.0 wt %, more preferably 8.0 to 10.0 wt %, particularly 8.5 to 9.5 wt %, and especially 8.7 to 9.1 wt %, based on the total weight of the mixture.

In one embodiment, the concentration of alkyl polyglycoside in the crude reaction product mixture is suitably in the range from 12.0 to 25.0 wt %, preferably 14.0 to 22.0 wt %, more preferably 16.0 to 20.0 wt %, particularly 17.0 to 19.0 wt %, and especially 17.5 to 18.5 wt %, based on the total weight of the mixture.

In one embodiment, the concentration of alkyl polyglycoside in the crude reaction product mixture is suitably in the range from 20.0 to 40.0 wt %, preferably 25.0 to 38.0 wt %, more preferably 29.0 to 36.0 wt %, particularly 31.0 to 35.0 wt %, and especially 32.0 to 34.0 wt %, based on the total weight of the mixture.

In one embodiment, the ratio by weight of alkyl polyglycoside product to alkyl glycoside starting material in the enzyme reaction according to the present invention is in the range from 1.1 to 3.5:1.0, suitably 1.2 to 3.0:1.0, preferably 1.4 to 2.5:1.0, more preferably 1.5 to 2.2:1.0, particularly 1.6 to 2.0:1.0, and especially 1.7 to 1.9:1.0.

In one embodiment, the alkyl chain component of the alkyl polyglycoside reaction product obtained using the process of the present invention suitably reflects/essentially is the same as the alkyl chain component of the alkyl glycoside starting material, i.e. all the definitions and ranges included herein defining the alkyl chain of the alkyl glycoside also apply to the alkyl polyglycoside reaction product.

In one embodiment, the alkyl chain of the alkyl polyglycoside, preferably alkyl polyglucoside, product comprises, consists essentially of, or consists of a mixture of C12 and C14 alkyl groups, suitably wherein the molar ratio of C12:C14 alkyl groups is in the range from 0.5 to 15:1.0, preferably 1.5 to 8:1.0, more preferably 2.0 to 4.0:1.0, particularly 2.6 to 3.4:1.0, and especially 2.9 to 3.1:1.0.

The chemical composition of glycoside component in the alkyl polyglycoside reaction product will depend upon the chemical composition of both the alkyl glycoside and the glycosyl donor. In one embodiment, the chemical composition of the glycoside component of the alkyl glycoside and glycosyl donor are the same, preferably both comprising, consisting essentially of, or consisting of glucose residues. Thus, the chemical composition of glycoside component of the alkyl polyglycoside preferably comprises, consists essentially of, or consists of glucose residues.

The mean length or mole-average degree of polymerization (mean DP) of the glycoside chain of the alkyl polyglycoside, preferably alkyl polyglucoside, reaction product, measured as described herein, is (i) suitably greater than or equal to 1.5, more suitably greater than or equal to 1.8, preferably greater than or equal to 2.1, more preferably greater than or equal to 2.4, particularly greater than or equal to 2.7, and especially greater than or equal to 2.9 glycoside units, preferably glucose units; and/or (ii) suitably less than or equal to 10.0, more suitably less than or equal to 8.0, preferably less than or equal to 6.0, more preferably less than or equal to 5.0, particularly less than or equal to 4.0, and especially less than or equal to 3.5 glycoside units, preferably glucose, units, i.e. per alkyl chain.

In one embodiment, the mean DP of the glycoside chain of the alkyl polyglycoside, preferably alkyl polyglucoside, reaction product is suitably in the range from 2.0 to 3.9, preferably 2.3 to 3.6, more preferably 2.6 to 3.4 particularly 2.8 to 3.2, and especially 2.9 to 3.0 glycoside units, preferably glucose units, i.e. per alkyl chain.

In one embodiment, the mean DP of the glycoside chain of the alkyl polyglycoside, preferably alkyl polyglucoside, reaction product is suitably in the range from 1.6 to 3.0, preferably 1.7 to 2.7, more preferably 1.8 to 2.4 particularly 1.9 to 2.3, and especially 2.0 to 2.2 glycoside units, preferably glucose units, i.e. per alkyl chain.

In one embodiment, the mean DP of the glycoside chain of the alkyl glycoside is increased during the process of the present invention to form the alkyl polyglycoside reaction product by (i) greater than or equal to 0.3, suitably greater than or equal to 0.5, more suitably greater than or equal to 1.0, preferably greater than or equal to 1.4, more preferably greater than or equal to 1.6, particularly greater than or equal to 1.8, and especially greater than or equal to 1.9 glycoside units, preferably glucose units; and/or (ii) less than or equal to 8.0, suitably less than or equal to 7.0, more suitably less than or equal to 6.0, preferably less than or equal to 5.0, more preferably less than or equal to 4.0, particularly less than or equal to 3.0, and especially less than or equal to 2.5 glycoside units, preferably glucose, units, i.e. per alkyl chain.

In one embodiment, the mean DP of the glycoside chain of the alkyl glycoside is increased to form the alkyl polyglycoside reaction product by an amount suitably in the range from 1.1 to 2.5, preferably 1.3 to 2.2, more preferably 1.5 to 2.0 particularly 1.6 to 1.9, and especially 1.7 to 1.8 glycoside units, preferably glucose units, i.e. per alkyl chain.

In one embodiment, the mean DP of the glycoside chain of the alkyl glycoside is increased to form the alkyl polyglycoside reaction product by an amount suitably in the range from 0.3 to 2.0, preferably 0.5 to 1.5, more preferably 0.7 to 1.3 particularly 0.9 to 1.1, and especially 0.95 to 1.05 glycoside units, preferably glucose units, i.e. per alkyl chain.

The aforementioned mean DP ranges suitably apply to alkyl polyglycoside reaction products formed from alkyl glycoside starting materials comprising alkyl chains as defined herein.

The alkyl polyglycoside, preferably alkyl polyglucoside, reaction product suitably comprises a composition or mixture of glycoside, preferably glucoside, chains comprising from 1 up to 10 glycoside units, i.e. selected from the group consisting of 1 (alkyl monoglycoside (DP1)), 2 (alkyl diglycoside (DP2)), 3 (alkyl triglycoside (DP3)), 4 (alkyl tetraglycoside (DP4)), 5 (alkyl pentaglycoside (DP5)), 6 (alkyl hexaglycoside (DP6)), 7 (alkyl heptaglycoside (DP7)), 8 (alkyl octaglycoside (DP8)), 9 (alkyl nonaglycoside (DP9)), 10 (alkyl decaglycoside (DP10)), and mixtures thereof. Alkyl polyglycosides having glycoside chains greater than 10 glycoside units (e.g. DP11 up to DP15) may also be present in the mixture, but these will generally be in smaller amounts.

In one embodiment, the alkyl polyglycoside, preferably alkyl polyglucoside, composition (i) suitably comprises less than 0.70, preferably less than 0.60, more preferably less than 0.50, particularly less than 0.45, and especially less than 0.40 mole fraction of DP1, and/or (ii) suitably comprises greater than 0.10, preferably greater than 0.15, more preferably greater than 0.20, particularly greater than 0.25, and especially greater than 0.30 mole fraction of DP1, both based on the total amount of DP1 to DP15 alkyl polyglycosides in the composition.

In one embodiment the alkyl polyglycoside, preferably alkyl polyglucoside, composition suitably comprises in the range from 0.19 to 0.51, preferably 0.23 to 0.47, more preferably 0.26 to 0.44, particularly 0.29 to 0.41, and especially 0.32 to 0.38 mole fraction of DP1, based on the total amount of DP1 to DP15 alkyl polyglycosides in the composition.

In one embodiment the alkyl polyglycoside, preferably alkyl polyglucoside, composition suitably comprises in the range from 0.30 to 0.68, preferably 0.37 to 0.64, more preferably 0.42 to 0.60, particularly 0.46 to 0.56, and especially 0.48 to 0.54 mole fraction of DP1, based on the total amount of DP1 to DP15 alkyl polyglycosides in the composition.

In one embodiment, the mole fraction of the alkyl monoglycoside (DP1) component of the alkyl polyglycoside reaction product and compositions defined herein is greater than the mole fraction of any other individual alkyl polyglycoside component present, e.g. D2, D3, D4 etc. up to D15.

The DP1 component of the alkyl polyglycoside reaction product may have the same chemical structure as at least some of the alkyl glycoside starting material, i.e. it could be considered to be unreacted starting material, but without being bound by theory, it is likely that substantially all of the alkyl monoglycoside starting material will have been extended by addition of glycoside residues in a coupling reaction and subsequently shortened by removal of glycoside residues in disproportionation and hydrolysis reactions during the process of the present invention to form DP1, DP2, DP3 and other components with longer glycoside chains.

In one embodiment, the alkyl polyglycoside, preferably alkyl polyglucoside, composition (i) suitably comprises less than or equal to 0.50, more suitably less than or equal to 0.45, preferably less than or equal to 0.40, more preferably less than or equal to 0.35, particularly less than or equal to 0.30, and especially less than or equal to 0.25 mole fraction of DP2, and/or (ii) suitably comprises greater than or equal to 0.05, more suitably greater than or equal to 0.10, preferably greater than or equal to 0.14, more preferably greater than or equal to 0.16, particularly greater than or equal to 0.18, and especially greater than or equal to 0.20 mole fraction of DP2, both based on the total amount of DP1 to DP15 alkyl polyglycosides in the composition.

In one embodiment, the alkyl polyglycoside, preferably alkyl polyglucoside, composition (i) suitably comprises less than or equal to 0.40, more suitably less than or equal to 0.30, preferably less than or equal to 0.25, more preferably less than or equal to 0.20, particularly less than or equal to 0.17, and especially less than or equal to 0.15 mole fraction of DP3, and/or (ii) suitably comprises greater than or equal to 0.02 more suitably greater than or equal to 0.05, preferably greater than or equal to 0.07, more preferably greater than or equal to 0.09, particularly greater than or equal to 0.11, and especially greater than or equal to 0.13 mole fraction of DP3, both based on the total amount of DP1 to DP15 alkyl polyglycosides.

In one embodiment, the alkyl polyglycoside, preferably alkyl polyglucoside, composition (i) suitably comprises less than 0.45, preferably less than 0.40, more preferably less than 0.35, particularly less than 0.30, and especially less than 0.26 mole fraction of DP4 to DP10, and/or (ii) suitably comprises greater than 0.05, preferably greater than 0.10, more preferably greater than 0.15, particularly greater than 0.18, and especially greater than 0.21 mole fraction of DP4 to DP10, both based on the total amount of DP1 to DP15 alkyl polyglycosides.

In one embodiment, the alkyl polyglycoside, preferably alkyl polyglucoside, composition (i) suitably comprises less than 0.15, preferably less than 0.10, more preferably less than 0.06, particularly less than 0.03, and especially less than 0.02 mole fraction of DP11 to DP15, and/or (ii) suitably comprises greater than 0.001, preferably greater than 0.002, more preferably greater than 0.005, particularly greater than 0.01, and especially greater than 0.015 mole fraction of DP11 to DP15, both based on the total amount of DP1 to DP15 alkyl polyglycosides.

In one embodiment, (i) the ratio of the mole fraction of DP1 to DP2 in the alkyl polyglycoside, preferably alkyl polyglucoside, composition is (a) suitably greater than 1.1:1.0, preferably greater than 1.5:1.0, more preferably greater than 1.8:1.0, particularly greater than 2.0:1.0, and especially greater than 2.2:1.0; and/or (b) suitably less than 3.5:1.0, preferably less than 3.0:1.0, more preferably less than 2.7:1.0, particularly less than 2.5:1.0, and especially less than 2.3:1.0; and/or (ii) the ratio of the mole fraction of DP2 to DP3 in the alkyl polyglycoside, preferably alkyl polyglucoside, composition is (a) suitably greater than 1.2:1.0, preferably greater than 1.3:1.0, more preferably greater than 1.6:1.0, particularly greater than 1.8:1.0, and especially greater than 2.0:1.0; and/or (b) suitably less than 3.3:1.0, preferably less than 2.9:1.0, more preferably less than 2.6:1.0, particularly less than 2.4:1.0, and especially less than 2.2:1.0; and/or (iii) the ratio of the mole fraction of DP1 to DP3 in the alkyl polyglycoside, preferably alkyl polyglucoside, composition is (a) suitably greater than 2.0:1.0, preferably greater than 3.0:1.0, more preferably greater than 4.0:1.0, particularly greater than 4.3:1.0, and especially greater than 4.6:1.0; and/or (b) suitably less than 7.5:1.0, preferably less than 6.5:1.0, more preferably less than 5.5:1.0, particularly less than 5.1:1.0, and especially less than 4.8:1.0.

In one embodiment, (i) the ratio of the mole fraction of DP1 to DP2 in the alkyl polyglycoside, preferably alkyl polyglucoside, composition is suitably in the range from 1.1 to 2.5:1.0, preferably 1.2 to 2.0:1.0, more preferably 1.3 to 1.8:1.0, particularly 1.4 to 1.6:1.0, and especially 1.5 to 1.55:1.0; and/or (ii) the ratio of the mole fraction of DP2 to DP3 in the alkyl polyglycoside, preferably alkyl polyglucoside, composition is suitably in the range from 1.1 to 2.5:1.0, preferably 1.2 to 2.0:1.0, more preferably 1.3 to 1.8:1.0, particularly 1.4 to 1.6:1.0, and especially 1.5 to 1.55:1.0; and/or (iii) the ratio of the mole fraction of DP1 to DP3 in the alkyl polyglycoside, preferably alkyl polyglucoside, composition is suitably in the range from 1.5 to 4.0:1.0, preferably 1.8 to 3.5:1.0, more preferably 2.0 to 3.0:1.0, particularly 2.2 to 2.5:1.0, and especially 2.3 to 2.4:1.0.

In one embodiment, the alkyl polyglycoside, preferably alkyl polyglucoside, reaction product or composition is in the form of a Flory-Schulz distribution. It is surprising for such a distribution to be obtained from an enzyme reaction as described herein.

The alkyl polyglycoside reaction product can be represented by the formula Rₚ-G_{q},

wherein
R is an alkyl group comprising p carbon atoms, both defined herein,
G is at least one monosaccharide residue, defined herein,
q is the number of monosaccharide residues,
q is (n+s) wherein n is the number of monosaccharide residues in the alkyl glycoside starting material and s is the increase in the number of monosaccharide residues that occurs during the enzyme reaction,
the mean value of n is defined herein (mean DP of starting material),
the mean value of q is defined herein (mean DP of reaction product), and
the mean value of s is defined herein (increase in mean DP that occurs during the enzyme reaction).

In one embodiment, the crude enzyme reaction product mixture comprises linear oligosaccharides which can be considered to be breakdown products of the glycosyl donor. The linear oligosaccharides preferably comprise, consist essentially of, or consist of glucose residues.

In one embodiment, the mean length or mole-average degree of polymerization (mean DP) of the linear oligosaccharides, measured as described herein, is suitably in the range from 1.5 to 6.0, preferably 2.0 to 5.0, more preferably 2.5 to 4.0, particularly 2.9 to 3.6, and especially 3.1 to 3.4.

In one embodiment, the concentration of linear oligosaccharides in the crude reaction product mixture is suitably in the range from 0.01 to 12.0 wt %, preferably 0.1 to 10.0 wt %, more preferably 0.2 to 8.0 wt %, particularly 1.0 to 6.0 wt %, and especially 3.0 to 5.0 wt %, based on the total weight of the mixture.

The concentration of linear oligosaccharides in the crude reaction product mixture having a degree of polymerization (DP) of 1 to 8 is suitably in the range from 0.005 to 8.0 wt %, preferably 0.05 to 6.0 wt %, more preferably 0.1 to 4.0 wt %, particularly 0.5 to 3.0 wt %, and especially 1.0 to 2.5 wt % based on the total weight of the mixture.

In one embodiment, the crude enzyme reaction product mixture comprises cyclodextrin, suitably comprising, consisting essentially of, or consisting of α-, β-, and γ-cyclodextrin, and preferably α- and β-cyclodextrin. The cyclodextrin may be unreacted cyclodextrin when cyclodextrin is used as the glycosyl donor and/or cyclodextrin produced as a side-product of the enzyme reaction with glycosyl donor defined herein other than cyclodextrin.

In one embodiment, the concentration in the crude reaction product mixture of cyclodextrin is less than 15.0 wt %, suitably less than 10.0 wt %, more suitably less than 7.0 wt %, preferably less than 5.0 wt %, more preferably less than 4.0 wt %, particularly less than 3.0 wt %, and especially less than 2.0 wt %, based on the total weight of the mixture.

In one embodiment, the concentration in the crude reaction product mixture of cyclodextrin is in the range from 0.5 to 25.0 wt %, suitably 1.0 to 20.0 wt %, preferably 3.0 to 15.0 wt %, more preferably 4.0 to 12.0 wt %, particularly 4.5 to 11.0 wt %, and especially 5.0 to 10.0 wt %, based on the total weight of the mixture.

In one embodiment, the weight ratio of α-cyclodextrin to β-cyclodextrin in the crude reaction product mixture is in the range from 0.1 to 5.0:1.0, suitably 0.2 to 4.0:1.0, preferably 0.5 to 2.0:1.0, more preferably 0.9 to 1.5:1.0, particularly 1.0 to 1.4:1.0, and especially 1.1 to 1.3:1.0.

In one embodiment, the weight ratio of α-cyclodextrin to β-cyclodextrin in the crude reaction product mixture is in the range from 0.1 to 3.0:1.0, suitably 0.15 to 2.0:1.0, preferably 0.2 to 1.0:1.0, more preferably 0.25 to 0.7:1.0, particularly 0.3 to 0.5:1.0, and especially 0.35 to 0.40:1.0.

For some applications, the crude reaction product mixture may be employed, but generally further processing/concentration/purification will be required. The reaction product mixture may be dewatered by evaporation, filtration or centrifugation, or dried by spray drying. The alkyl polyglycoside, preferably alkyl polyglycoside, reaction product may be purified by a variety of methods including chromatography, precipitation, adsorption, filtration and centrifugation, as known in the art. In particular, membrane filtration or flash chromatography may be used to produce a purified alkyl polyglycoside composition.

In one embodiment, a purified alkyl polyglycoside composition comprises, consists essentially of, or consists of (i) suitably in the range from 6.0 to 60.0 wt %, preferably 15.0 to 45.0 wt %, more preferably 21.0 to 39.0 wt %, particularly 27.0 to 33.0 wt %, and especially 29.0 to 31.0 wt %, of alkyl polyglycoside defined herein; and (ii) suitably in the range from 40.0 to 94.0 wt %, preferably 55.0 to 85.0 wt %, more preferably 61.0 to 79.0 wt %, particularly 67.0 to 73.0 wt %, and especially 69.0 to 71.0 wt %, of water, both based on the total weight of the composition.

In one embodiment, a purified alkyl polyglycoside composition comprises, consists essentially of, or consists of (i) preferably in the range from 45.0 to 55.0 wt %, more preferably 50.0 to 52.0 wt %, of alkyl polyglycoside defined herein; and (ii) preferably in the range from 45.0 to 55.0 wt %, more preferably 48.0 to 50.0 wt %, of water, both based on the total weight of the composition.

The alkyl polyglycosides, preferably alkyl polyglucosides, defined herein are suitable to be used as an emulsifier, solubilizer, foamer, cleanser and/or thickener preferably in personal care formulations, for example in oil-in-water or water-in-oil emulsions. The alkyl polyglycosides are particularly effective as foaming agents and may improve the amount, stability and/or quality of the foam formed. The alkyl polyglycosides may be used in any of the forms described herein, i.e. in crude or purified enzyme reaction product.

The alkyl polyglycosides defined herein, particularly in pure form, are suitable for use as emulsifiers in personal care applications. Personal care emulsion products can take the form of creams and milks desirably and typically include emulsifier to aid formation and stability of the emulsion. Suitably, personal care emulsion products use emulsifiers in amounts in the range from about 1 to about 20% by weight, preferably 3 to 6% by weight of the emulsion. The alkyl polyglycosides may also be combined with other emulsifiers and emulsion stabilisers in emulsions. Examples of such emulsifiers include non-ionic emulsifying waxes, for example fatty alcohols and polyol esters.

The oil-in-water or water-in-oil emulsions comprising alkyl polyglycosides may include various other personal care ingredients for example one or more of such as cleaning agents, hair conditioning agents, skin conditioning agents, hair styling agents, antidandruff agents, hair growth promoters, perfumes, sunscreen compounds, pigments, moisturizers, film formers, humectants, alpha-hydroxy acids, hair colours, make-up agents, detergents, thickening agents, antiseptic agents, deodorant actives and surfactants.

The oil phase of such emulsions are typically emollient oils of the type used in personal care or cosmetic products, which are oily materials which are liquid at ambient temperature or solid at ambient temperature, in bulk usually being a waxy solid, provided they are liquid at an elevated temperature, preferably up to 100° C. more preferably up to 80° C., so such solid emollients suitably have melting temperatures less than 100° C., and preferably less than 70° C., at which it can be included in and emulsified in the composition.

The concentration of the oil phase may vary widely and the amount of oil is suitably in the range from 1 to 90 wt %, preferably 3 to 60 wt %, more preferably 5 to 40 wt %, particularly 8 to 20 wt %, and especially 10 to 15 wt %, based on the total emulsion.

The amount of water present in the emulsion is suitably greater than 5 wt %, preferably in the range from 30 to 90 wt %, more preferably 50 to 90 wt %, particularly 70 to 85 wt %, and especially 75 to 80 wt %, based on the total emulsion. The amount of emulsifier, preferably alkyl polyglycosides defined herein, used in such emulsions is suitably in the range from 0.1 to 10 wt %, preferably 0.5 to 8 wt %, more preferably 1 to 7 wt %, particularly 1.5 to 6 wt %, and especially 2 to 5.5 wt %, based on the total emulsion.

The end use formulations of such emulsions include moisturizers, sunscreens, after sun products, body butters, gel creams, high perfume containing products, perfume creams, baby care products, hair conditioners, hair relaxer formulations, skin toning and skin whitening products, water-free products, anti-perspirant and deodorant products, tanning products, cleansers, 2-in-1 foaming emulsions, multiple emulsions, preservative free products, emulsifier free products, mild formulations, scrub formulations e.g. containing solid beads, silicone in water formulations, pigment containing products, sprayable emulsions, colour cosmetics, conditioners, shower products, foaming emulsions, make-up remover, eye make-up remover, and wipes. Such formulations include green formulations, natural formulations and naturally certified formulations.

A further use of such alkyl polyglycoside emulsifiers is to reduce irritation of primary surfactants such as alkyl ether sulphates and alkyl sulphates, for example in baby care formulations. End use formulations of such emulsions include mild and/or sulphate free detergents, microemulsions, cleansers including acne cleansers, shampoos including 2-in-1 with conditioners and baby shampoos, facial and body washes, shower gels and shower creams, hand soaps including cream hand soaps.

The alkyl polyglycosides may also be used as emulsifiers in oil-in-water or water-in-oil emulsions in health care applications. Examples include liquid emulsion oral treatments, medical shampoos, topical treatment creams, lotions and ointments, anti-acne treatment creams, lotions and tonics, suppositories.

The alkyl polyglycosides may also be used as thickening agents in detergent systems. Applications include mild detergents, sulphate free detergents, microemulsions, cleansers including acne cleansers, shampoos in general, baby shampoos and 2-in-1 shampoos and conditioners, facial and body washes, shower creams and gels, hand soaps. Suitably the alkyl polyglycosides thickening agent is present in the range from of 1.0 to 5.0 wt % in the detergent system.

The alkyl polyglycosides may also be used as an effective solubilizer, to solubilize a wide range of compounds that are insoluble or poorly soluble in water. Such compounds may be active ingredients or solutes, such as lipids, surfactants, particularly non-ionic surfactants, perfumes, essential oils, colorants, pigments, proteins, steroids, and active pharmaceutical ingredients (APIs). The water insoluble material is suitably an active cosmetic, personal care or pharmaceutical ingredient.

In one embodiment, the alkyl polyglycosides can be used as a solubilizing agent for the personal care and health care sectors. In these sectors the most commonly used solubilising agents generally contain at least one non-renewable polyoxyethylenated derivative. For example, polysorbate 20 and polysorbate 80 are often used in cosmetic and pharmaceutical compositions. Alternatives to polyethoxylated solubilizing agents (e.g. conventional alkyl glycosides) do not generally have an equivalent level of effectiveness. The alkyl polyglycosides defined herein can outperform existing alkyl glycosides and ethoxylated surfactants, such as polysorbate 20 and 80, as solubilizers. Thus, the alkyl polyglycosides can be used as a complete, or partial, replacement for polyoxyethylenated derivatives and conventional alkyl glycosides, in cosmetic, personal care and health care compositions, particularly for replacing polysorbates.

Suitable health care applications where the alkyl polyglycosides may be used as solubilizers include liquid emulsion oral treatments, medical shampoos, topical treatment creams, lotions, ointments and cleansing wipes, anti-acne treatment creams, lotions and tonics.

In personal care formulations, the solubilizers are key components of aqueous based systems that incorporate oily components such as perfumes, essential oils, lipophilic actives, oily vitamins and emollient oils. Pre-solubilization of these oily components into the personal care formulations ensures an acceptable clear product. Typical products that can benefit from the use of the solubilizers include clear shampoos, sulphate free shampoos, clear combined shampoos and conditioners, clear conditioners, clear facial washes, clear shower gels and bath foams, clear hair and skin gels, aqueous/alcoholic hair spritzes, aqueous/alcoholic body sprays, aftershaves, colognes, skin cleansers and toners, make up removers, anti-bacterial wipes, lotions, ointments and gels, general wet wipes. Such formulations include green formulations, natural formulations and naturally certified formulations.

The personal care composition may also contain other materials such excipients that are soluble in water, in particular non-ionic, anionic, cationic surfactants, salts, pH adjusters, hydrating agents, chelates, metal ions, polymers, dispersing agents, colorants, preservatives and hydrotropes.

In one embodiment, a personal care composition comprises (i) in the range from 0.001 to 10.0 wt %, preferably 0.005 to 5.0 wt %, more preferably 0.01 to 3.0 wt %, particularly 0.05 to 2.0 wt %, and especially 0.1 to 1.0 wt % of at least one active ingredient, (ii) in the range from 0.01 to 75.0 wt %, preferably 0.05 to 50.0 wt %, more preferably 0.1 to 30.0 wt %, particularly 0.5 to 20.0 wt %, and especially 0.1 to 10.0 wt % of alkyl polyglycosides defined herein, and/or (iii) in the range from 15.0 to 99.99 wt %, preferably 45.0 to 99.95 wt %, more preferably 67.0 to 99.89 wt %, particularly 78.0 to 99.45 wt %, and especially 89.0 to 98.9 wt % of water.

All of the features described herein may be combined with any of the above aspects, in any combination.

The following test methods were used;

1) Composition of Alkyl Polyglycoside.

The alkyl polyglycosides were analysed using an HPLC system with a C-18 column. An appropriate dilution of the sample was injected on the system and separated on the column using a dual mobile phase. The mobile phase had a hydrophobic component (e.g. acetonitrile) and a hydrophilic component (e.g. 0.1% acetic acid). The method started with a low hydrophobic content in the mobile phase which was gradually increased during the analysis. To identify and quantify the components eluting from the column a CAD-detector (Charged Aerosol Detector) and a mass spectrometer were used. In order to get a constant composition of mobile phase to the detector, a reversed gradient was used which connected to the analytical flow just before the detector. This method makes it possible to calculate the DP profile, including the mean DP, of the alkyl polyglycoside and the glycoside (or glucose) units conversion rate of the enzyme reaction.

2) Composition of Linear Oligosaccharides.

Linear oligosaccharides were analysed on an Anion-Exchange Chromatography (HPAEC) system using a Dionex CarboPac PA200 column and two guard columns.

Samples were diluted to an appropriate concentration in a suitable solvent and injected on the system and separated on the column using a mobile phase with gradually increasing sodium acetate concentration in 100 mM NaOH. To identify the components eluting from the column a Dionex ED40 Electrochemical Detector was used. For quantification, appropriate standards were analysed and quantities of the respective species were determined from the standard curve. This method makes it possible to calculate the DP profile, including the mean DP, of the linear oligosaccharides.

3) Composition of Cyclodextrin.

Cyclodextrin was analysed using the same basic method as described above for linear oligosaccharides analysis.

4) Foam Measurement

Foaming was evaluated using a Dynamic Foam Analyser from Kruss. A volume of 50 ml of 1.5 wt % alkyl polyglucoside solution was added to the cylinder. The solution was stirred at 3,000 rpm for 2 minutes, and the foam height measured. PEG-80 sorbitan laurate and cocamidopropyl betaine were used as surfactant controls.

5) Cholesterol Solubility

a) Maximum Solubility Sample Preparation.

15 wt % solution was prepared by dissolving alkyl polyglucoside in distilled water. 35 mg of cholesterol was dosed into pre-weighed Eppendorf tubes. 1 ml of each solution was pipetted into the Eppendorf tubes and agitated for 5 seconds using a vortex mixer, and left to equilibrate on a roller at 60 rpm for 1 hour. After mixing, each tube was subjected to centrifugation for 15 minutes at 14,800 rpm and 20° C. The supernatant was syringed out and transferred to a second Eppendoft tube, using a 1 ml syringe with a 0.22 μm nylon filter. The supernatant was then centrifuged for a second time at 14,800 rpm. 50 μl of the resulting supernatant was added to 950 μl of methanol in an HPLC vial and assayed by HPLC. PEG-80 sorbitan laurate, NatraGem™ S140 (ex Croda) and decyl glucoside were used as surfactant controls.

b) Preparing Standard Curve.

A stock solution of cholesterol (10 mg) in methanol (10 ml) was prepared. The solution was agitated using a vortex mixer for 10 seconds to ensure complete dissolution of the cholesterol to give a solution with 1.00 mg ml⁻¹ concentration of cholesterol. Six calibration samples were prepared by dilution of the stock solution, with concentrations of 0.050, 0.025, 0.020, 0.010, 0.0050 and 0.0010 mg ml⁻¹. The peak areas obtained in the analysis of the calibration samples were used to determine the calibration curve.

c) HPLC Conditions.

| HPLC column: | Zorvax Eclipse Plus C18, 5 μm, 5 cm | |
|---|---|---|
| Mobile phase composition: | 95/5 methanol/water (isocratic) | |
| Column temperature: | 30° | C. |
| Flow rate: | 1.0 | ml min⁻¹ |
| Injection volume: | 5.0 | μl |
| Run time: | 10 | mins |
| Detection wavelength: | 204 | nm |

6) Essential Oils Solubility.

13 wt % solution of alkyl polyglucoside in distilled water was prepared and separately mixed with lemon oil, lavender oil, tea tree oil and patchouli oil at a ratio of 1:7 (w/w), and then the mixtures were added dropwise under stirring to water to a final concentration of 1 wt % essential oil and 7 wt % alkyl polyglucoside. The mixtures were stirred with a magnetic bar for one hour and no oil drops were observed, dispersed in solution or on the surface of any of the mixtures, indicating complete solubilization of the essential oil in all cases. The mixtures were then left at room temperature for 5 hours and observed for phase separation. Lauryl glucoside was used as a surfactant control with both lemon oil and lavender oil.

### EXAMPLES

### Example 1

31 kg of maltodextrin having a DE value of 1 was charged to 39 kg of water in a reaction vessel at room temperature and the reaction mixture was agitated with stirring until the maltodextrin was dissolved. The maltodextrin solution was then drummed off. 10 kg of 50 wt % aqueous solution of lauryl glucoside was charged to 20 kg of water in the reaction vessel as a free-flowing liquid (at approx. 50° C.) and the stirring speed adjusted to avoid excessive foaming. HCl solution was charged to the reaction vessel to adjust the pH to 6.5. The previously prepared maltodextrin solution was then charged to the reaction vessel with stirring. The temperature was increased to 65° C. and stirrer speed adjusted to avoid excessive foaming. When the temperature had reached 65° C., 0.5 kg of Thermoanaerobacter sp CGTase (EC 2.4.1.19) enzyme preparation (equivalent to 17 KNU-CP per kg of reaction mixture) was added to the reaction vessel. The reaction mixture was stirred and the reaction continued for 20 hours. The reaction mixture was then heated to 95° C., kept constant at this temperature for 2.5 hours to inactivate the enzyme, cooled to 40-50° C. with stirring and the pH adjusted to 11.5 using 50% sodium hydroxide solution. A sample of the crude reaction mixture was taken, subjected to the test procedures described herein, and exhibited the following properties;

a) Reaction mixture:
i) Alkyl polyglucoside=6.2 wt %.
ii) α-Cyclodextrin=2.3 wt %.
iii) β-Cyclodextrin=3.4 wt %.
iv) Linear oligosaccharides=4.6 wt %.
v) Foam height=130 mm (PEG-80 sorbitan laurate=92, cocamidopropyl betaine=78).

b) Alkyl polyglucoside:
i) DP1=0.35 mole fraction.
ii) DP2=0.23 mole fraction.
iii) DP3=0.15 mole fraction.
iv) DP4 to DP10=0.25 mole fraction.
v) DP11 to DP15=0.02 mole fraction.
vi) Mean DP=2.9 glucose units.
vii) Increase in mean DP=1.8 glucose units.
viii) Glucose conversion=12.5 wt %.

c) Linear oligosaccharides:
i) DP1 to DP8=2.6 wt %.
ii) DP9 and above=2.0 wt %.
iii) Mean DP=3.0 glucose units.

The remaining reaction mixture was left in the reaction vessel without stirring at room temperature for at least 24 hours until precipitation occurred. After precipitation had completed, the precipitate was collected by separating the solid phase from the solution by centrifugation. The liquid supernatant was then subjected to flash chromatography to remove any residual sugar component, including linear oligosaccharides and cyclodextrins, using an Isolera system from Biotage equipped with a Snap Ultra C18 column (Biotage) with a volume of 654 ml. The supernatant was diluted with 99.5% ethanol to a final ethanol concentration of 20 wt % and then loaded on the column which had been pre-equilibrated with 20% ethanol. The column was then washed with equilibration solution to remove unbound sugars and protein from the column. The alkyl polyglucoside was then eluted by a step-wise change of mobile phase to 80% ethanol, followed by a final step with 99.5% ethanol. Most of the product was recovered in the 80% ethanol step and all alkyl polyglucoside containing fractions were pooled. The ethanol from the pooled fractions was removed by evaporation in a Büchi rotavapor and the remaining water was removed by freeze drying to produce a white, free-flowing powder of alkyl polyglucoside reaction product, which was subjected to the test procedures described herein, and exhibited the following properties;
i) DP1=0.35 mole fraction.
ii) DP2=0.23 mole fraction.
iii) DP3=0.18 mole fraction.
iv) DP4 to DP10=0.23 mole fraction.
v) DP11 to DP15=0.002 mole fraction.
vi) Mean DP=3.0 glucose units.

### Example 2

100 grams of a 50 wt % solution of lauryl glucoside and 300 grams of maltodextrin having a DE value of 6 were mixed with 0.6 litres of water in a 2-litre reaction vessel with top stirring. The pH was adjusted to 7 with HCl and the temperature was then increased to 45° C. When the temperature had reached 45° C., 2.0 g of Bacillus macerans CGTase (EC 2.4.1.19) enzyme preparation (equivalent to 2400 U per kg of reaction mixture) was added and the reaction allowed to proceed at constant temperature for 24 hours. The reaction mixture was heated to 95° C. for 2 hours to inactivate the enzyme, and then cooled to ambient temperature. The reaction mixture was then subjected to flash chromatography to remove any residual sugar component using a C18-Silica column (400 g, Biotage). The entire reaction mixture was diluted with 99.5% ethanol to a final concentration of 20% ethanol, before loading on the column, previously equilibrated with 20% ethanol. The column was washed with 16 column volumes of 20% ethanol for removal of the residual sugar. 6 column volumes of 40% ethanol were then applied for elution of the bound enzyme. Finally, the alkyl polyglycoside reaction product was eluted with 80% ethanol and concentrated by means of freeze drying, yielding a white free-flowing powder. The resulting alkyl polyglucoside reaction product was subjected to the test procedures described herein, and exhibited the following properties;
i) DP1=0.38 mole fraction.
ii) DP2=0.25 mole fraction.
iii) DP3=0.14 mole fraction.
iv) DP4 to DP10=0.23 mole fraction.
v) Mean DP=2.6 glucose units.
vi) Increase in mean DP=1.5 glucose units.
vii) Glucose conversion=10.8 wt %.
viii) Cholesterol Solubility=17.8 mg ml⁻¹ (PEG-80 sorbitan laurate=0.7 mg ml⁻¹, NatraGem™ S140=3.5 mg ml⁻¹, decyl glucoside=5.2 mg ml⁻¹).

### Example 3

1.9 kg of a 50 wt % aqueous solution of lauryl glucoside was mixed with 1.6 kg of water at 50° C. under constant stirring in a reaction vessel. Another 3.4 kg of water was added and the temperature was increased to 65° C. 1.9 kg of α-cyclodextrin was gradually added together with 1.0 kg of water, and the reaction mixture was agitated with stirring for 30 minutes until all components were dissolved. HCl solution was charged to the reaction vessel to adjust the pH to 7.0. 0.1 kg of Thermoanaerobacter sp CGTase (EC 2.4.1.19) enzyme preparation (equivalent to 34 KNU-CP per kg of reaction mixture) was then added to the reaction mixture which was agitated and the reaction continued for 5 hours. The reaction mixture was then heated to 95° C., kept constant at this temperature for 3 hours to inactivate the enzyme, cooled to 40-50° C. with stirring and the reaction mixture recovered from the reaction vessel. The alkyl polyglucoside reaction product was subjected to the test procedures described herein, and exhibited the following properties;

a) Reaction mixture:
i) Alkyl polyglucoside=17.8 wt %.
ii) α-Cyclodextrin=5.5 wt %.
iii) β-Cyclodextrin=4.7 wt %.
iv) Linear oligosaccharides=0.3 wt %.

b) Alkyl polyglucoside:
i) DP1=0.38 mole fraction.
ii) DP2=0.21 mole fraction.
iii) DP3=0.15 mole fraction.
iv) DP4 to DP10=0.24 mole fraction.
v) DP11 to DP15=0.02 mole fraction.
vi) Mean DP=2.9 glucose units.
vii) Increase in mean DP=1.8 glucose units.
viii) Glucose conversion=45 wt %.

### Example 4

135.6 g of a 50 wt % aqueous solution of lauryl glucoside was mixed with 77.1 g of water in a 400 ml reaction vessel under stirring. To the solution was added 56 g of β-cyclodextrin undecahydrate powder and the reaction mixture was agitated with stirring until the 3-cyclodextrin dissolved. 30.9 ml of HCl solution was charged to the reaction vessel to adjust the pH to 6.0 and the temperature was raised to 75° C., after which 0.45 ml of an enzyme preparation containing Thermoanaerobactersp. CGTase (EC 2.4.1.19) was added to the reaction vessel. The reaction mixture was stirred and the reaction continued for 24 hours. The reaction mixture was then heated to 95° C., kept constant at this temperature for 2.5 hours to inactivate the enzyme and then cooled. A sample of the crude reaction mixture was taken, subjected to the test procedures described herein, and exhibited the following properties;

a) Reaction mixture:
i) Alkyl polyglucoside=33 wt %.
ii) α-Cyclodextrin=1.0 wt %.
iii) β-Cyclodextrin=2.6 wt %.
iv) Linear oligosaccharides=1.0 wt %.

b) Alkyl polyglucoside:
i) DP1=0.52 mole fraction.
ii) DP2=0.23 mole fraction.
iii) DP3=0.11 mole fraction.
iv) DP4 to DP10=0.13 mole fraction.
v) DP11 to DP15=0.003 mole fraction.
vi) Mean DP=2.1 glucose units.
vii) Increase in mean DP=1.0 glucose units.
viii) Glucose conversion=60 wt %.
ix) Foam height=108 mm (PEG-80 sorbitan laurate=92, cocamidopropyl betaine=78).

c) Linear oligosaccharides:
i) DP1 to DP8=0.9 wt %.
ii) DP9 and above=1.0 wt %.
iii) Mean DP=2.6

d) Essential oils solubility:

Lemon oil, lavender oil, tea tree oil and patchouli oil (1 wt %) mixtures with 7 wt % alkyl polyglucoside all remained clear with no phase separation after 5 hours (lemon oil and lavender oil mixtures were white and cloudy, and had phase separated after five hours using 7 wt % lauryl glucoside as solubilizer).

The above examples illustrate the improved properties of the process and compositions according to the present invention.

1. A process for the preparation of C4 to C24 alkyl polyglycosides by reacting with an enzyme a C4 to C24 alkyl glycoside and a glycosyl donor comprising monosaccharide residues, wherein (a) the reaction mixture comprises (i) monosaccharide residues in the glycosyl donor to alkyl glycoside at a molar ratio of less than 40.0:1.0, and optionally (ii) alkyl glycoside at greater than or equal to 1.0 wt %; (b) greater than or equal to 3.0 wt % of the monosaccharide residues in the glycosyl donor are transferred to the alkyl glycoside (glycoside units conversion); and (c) the reaction product comprises alkyl polyglycoside optionally comprising (i) greater than 0.10 mole fraction of alkyl monoglycoside (DP1), and/or (ii) a mole-average degree of polymerization (mean DP) of the glycoside chains of greater than or equal to 1.5 units.

2. A process of reacting with an enzyme;
(i) a glycosyl donor comprising monosaccharide residues; and
(ii) an alkyl glycoside of the formula Rₘ-Gₙ, wherein
R is an alkyl group comprising m carbon atoms,
m is 4 to 24,
G is at least one monosaccharide residue, and
n is the number of monosaccharide residues;
in a reaction mixture to form a reaction product comprising;
(iii) an alkyl polyglycoside of the formula Rₚ-G_{q},
wherein
R is an alkyl group comprising p carbon atoms, p is 4 to 24,
G is at least one monosaccharide residue,
q is the number of monosaccharide residues, and the mean value of q (mean DP) is greater than or equal to 1.5,
q=(n+s) wherein n is defined in (ii) and s is the increase in the number of monosaccharide residues that occurs during the enzyme reaction, and
the mean value of s is greater than or equal to 0.5; and
(iv) greater than or equal to 3.0 wt % of the monosaccharide residues in the glycosyl donor are transferred to the alkyl glycoside (glycoside units conversion) during the enzyme reaction.

3. The process according to claim 1 wherein the molar ratio of monosaccharide residues in the glycosyl donor to alkyl glycoside is less than 20.0:1.0.

4. The process according to claim 3 wherein the molar ratio of monosaccharide residues in the glycosyl donor to alkyl glycoside is less than 6.0:1.0.

5. The process according to claim 1 wherein the glycoside units conversion is greater than or equal to 15.0 wt %.

6. The process according to claim 5 wherein the glycoside units conversion is greater than or equal to 35.0 wt %.

7. The process according to claim 1 wherein the mean DP of the alkyl polyglycosides is greater than or equal to 1.8 units.

8. The process according to claim 7 wherein the mean DP of the alkyl polyglycosides is greater than or equal to 2.1 units.

9. The process according to claim 1 wherein the reaction product comprises less than 15.0 wt % cyclodextrin.

10. The process according to claim 9 wherein the reaction product comprises less than 5.0 wt % cyclodextrin.

11. The process according to claim 1 wherein the glycosyl donor is cyclodextrin, optionally b-cyclodextrin.

12. The process according to claim 1 wherein the reaction mixture comprises greater than or equal to 8.0 wt % alkyl glycoside.

13. The process according to claim 1 wherein the reaction mixture comprises greater than or equal to 10.0 wt % glycosyl donor.

14. The process according to claim 1 comprising at least one of (i) the molar ratio of monosaccharide residues in the glycosyl donor to alkyl glycoside is 0.8 to 2.5:1.0, and/or (ii) the glycoside units conversion is greater than or equal to 50.0 wt %, and/or (iii) the mean DP of the alkyl polyglycosides is 1.9 to 2.3 units, and/or (iv) the reaction product comprises less than 4.0 wt % cyclodextrin.

15. The process according to claim 14 comprising at least two of (i) to (iv).

16. The process according to claim 14 comprising at least three of (i) to (iv).

17. The process according to claim 14 comprising all of (i) to (iv).

18. A composition comprising C4 to C24 alkyl polyglycosides wherein the amount of alkyl monoglycoside (DP1) is greater than 0.10 mole fraction and the mole-average degree of polymerization (mean DP) of the glycoside chains is greater than or equal to 1.8 units.

19. The composition according to claim 18 wherein the mean DP is greater than or equal to 2.1 units.

20. The composition according to claim 18 wherein the amount of DP1 is 0.30 to 0.68 mole fraction.

21. The composition according to claim 18 wherein the amount of alkyl diglycoside (DP2) is 0.10 to 0.45 mole fraction.

22. (canceled)

23. (canceled)

24. (canceled)

25. (canceled)
